(19) United States
(12) Patent Application Publication
(10) Pub. No.: US 2025/0089835 A1
(43) Pub. Date: Mar. 20, 2025

(54) AVIATION HELMET

(71) Applicant: GENTEX CORPORATION, Simpson, PA (US)

(72) Inventor: MATTHEW HANUDEL, DEDHAM, MA (US)

(73) Assignee: GENTEX CORPORATION, Simpson, PA (US)

(21) Appl. No.: 18/559,599

(22) PCT Filed: May 27, 2022
(86) PCT No.: PCT/US2022/031260

§ 371 Date: Nov. 8, 2023

Related U.S. Application Data
(60) Provisional application No. 63/194,484, filed on May 28, 2021.

(51) Int. Cl. A42B 3/04; A42B 3/22; A61F 9/02

(52) U.S. Cl. CPC A42B 3/0406; A42B 3/228; A61F 9/022

(57) ABSTRACT

An adjustment mechanism is disclosed that includes a housing that has a sidewall extending circumferentially around a central axis defining an opening. A first aperture extends through the sidewall perpendicular to the central axis and a receiving member rotatably coupled to the housing and includes a protrusion that extends along the central axis and an aperture spaced from the central axis. A cable has a first end that extends through the first aperture of the housing and is fixedly coupled to the receiving member and a second end fixed relative to the housing. A knob is rotatably coupled to the housing and is configured to engage the receiving member such that rotation of the knob causes the receiving member to rotate. A locking element engages with the sidewall of the housing, and is configured to disengage the sidewall in response to a manual rotation of the knob by a user.

### CROSS-REFERENCE TO RELATED APPLICATIONS

This application claims the benefit of U.S. Provisional Patent Application No. 63/194,484 filed May 28, 2021 entitled “Aviation Helmet”, which is incorporated by reference herein in its entirety.

### TECHNICAL FIELD

The present invention generally relates to an aviation helmet and, more particularly, to an aviation helmet with an adjustable nape protection assembly, an adjustable visor, a visor cover assembly, and an adjustable mandible shield.

### SUMMARY

In one embodiment there is an adjustment mechanism including a housing including a sidewall extending circumferentially around a central axis, the sidewall defining an opening extending along the central axis and including a first aperture extending through the sidewall in a direction perpendicular to the central axis. The adjustment mechanism further includes a receiving member coupled to the housing and rotatable about the central axis, the receiving member including a protrusion extending along the central axis, the protrusion including an aperture extending along an axis spaced from and parallel to the central axis. The adjustment mechanism further includes a cable having a first end and a second end, the first end extending through the first aperture in the housing and the aperture in the receiving member and fixedly coupled to the receiving member, the second end fixed relative to the housing. The adjustment mechanism further includes a knob coupled to the housing and rotatable about the central axis, the knob including a first protrusion configured to engage the protrusion of the receiving member such that rotation of the knob causes the receiving member to rotate. The adjustment mechanism further includes a locking element engaged with an interior surface of the sidewall of the housing, the locking element configured to be disengaged with the interior surface of the sidewall in response to a manual rotation of the knob by a user.

In some embodiments, the locking element includes a biasing element engaged with the interior surface of the sidewall of the housing and configured to disengage from the interior surface of the sidewall in response to manual rotation of the knob by the user. In some embodiments, the biasing element is a spring coiled about the central axis, a first terminus extending inwardly toward the central axis, a second terminus, opposite the first terminus, extending from inwardly toward the central axis. In some embodiments, at least one of the first terminus and second terminus of the spring is configured to engage the first protrusion of the knob such that rotation of the knob causes the spring to compress. In some embodiments, the adjustment mechanism further includes a base configured to receive at least one compressible comfort pad, the base comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side.

In some embodiments, the adjustment mechanism further includes a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism. In some embodiments, the base includes a flexible arm configured to couple the base to a helmet shell, wherein rotation of the knob causes the base to be displaced relative to the housing and causes the flexible arm to be elastically deformed.

In another embodiment, there is an adjustment mechanism including a housing including a sidewall extending circumferentially around a central axis, the sidewall defining an opening extending along the central axis and including a first aperture extending through the sidewall in a direction perpendicular to the central axis. The adjustment mechanism further includes a receiving member coupled to the housing and rotatable about the central axis, the receiving member including a protrusion extending along the central axis, the protrusion including an aperture extending along an axis spaced from and parallel to the central axis. The adjustment mechanism further includes a cable having a first end and a second end, the first end extending through the first aperture in the housing and the aperture in the receiving member and fixedly coupled to the receiving member, the second end fixed relative to the housing. The adjustment mechanism further includes a knob coupled to the housing and rotatable about the central axis, the knob including a first protrusion configured to engage the protrusion of the receiving member such that rotation of the knob causes the receiving member to rotate. The adjustment mechanism further includes a locking element engaged with an interior surface of the sidewall of the housing, the locking element configured to be disengaged with the interior surface of the sidewall in response to a manual rotation of the knob by a user. The adjustment mechanism further includes a base configured to receive at least one compressible comfort pad, the base comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side. The adjustment mechanism further includes a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism. The locking element includes a biasing element engaged with the interior surface of the sidewall of the housing and configured to disengage from the interior surface of the sidewall in response to manual rotation of the knob by the user.

In some embodiments, the biasing element is a spring coiled about the central axis, a first terminus extending inwardly toward the central axis, a second terminus, opposite the first terminus, extending from inwardly toward the central axis. In some embodiments, at least one of the first terminus and second terminus of the spring is configured to engage the first protrusion of the knob such that rotation of the knob causes the spring to compress.

In another embodiment there is an aviation helmet including a helmet shell having an outer surface and an inner surface and a base configured to be positioned proximate a nape of a user's neck when the user wears the helmet shell, the base adjustably coupled to the helmet shell by an adjustment mechanism. The adjustment mechanism includes a housing fixedly coupled to the helmet shell and including a sidewall extending circumferentially around a central axis, the sidewall defining an opening extending along the central axis and including a first aperture extending through the sidewall in a direction perpendicular to the central axis. The adjustment mechanism further includes a receiving member coupled to the housing and rotatable about the central axis, the receiving member including a protrusion extending along the central axis, the protrusion including an aperture extending along an axis spaced from and parallel to the central axis. The adjustment mechanism further includes a cable coupled to the base and having a first end and a second end, the first end extending through the first aperture in the housing and the aperture in the receiving member and fixedly coupled to the receiving member, the second end fixedly coupled to the helmet shell. The adjustment mechanism further includes a knob coupled to the housing and rotatable about the central axis, the knob including a first protrusion configured to engage the protrusion of the receiving member such that rotation of the knob causes the receiving member to rotate. The adjustment mechanism further includes a locking element engaged with an interior surface of the sidewall of the housing, the locking element configured to be disengaged with the interior surface of the sidewall in response to a manual rotation of the knob by a user.

In some embodiments, the locking element includes a biasing element engaged with the interior surface of the sidewall of the housing and configured to disengage from the interior surface of the sidewall in response to manual rotation of the knob by the user. In some embodiments, the biasing element is a spring coiled about the central axis, a first terminus extending inwardly toward the central axis, a second terminus, opposite the first terminus, extending from inwardly toward the central axis. In some embodiments, at least one of the first terminus and second terminus of the spring is configured to engage the first protrusion of the knob such that rotation of the knob causes the spring to compress. In some embodiments, the base is configured to receive at least one compressible comfort pad, the base being comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side.

In some embodiments, the aviation helmet further includes a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism. In some embodiments, the base includes a flexible arm coupling the base to the helmet shell, and wherein rotation of the knob causes the base to be displaced relative to the housing and causes the flexible arm to be elastically deformed.

In another embodiment there is a visor assembly for a helmet, the visor assembly includes a lens sized to extend across at least a portion of a user's face, and a first arm coupled to the lens and rotatable about a first axis such that rotation of the first arm causes the lens to rotate about the first axis, the first arm having a first portion extending along a second axis, and a second portion extending from an end of the first portion at an obtuse angle from the second axis along a third axis such that the first arm forms a bent shape. The visor assembly further includes a second arm coupled to the second portion of the first arm and rotatable about the first axis, the second arm including a biasing element engaged with the second portion of the first arm and a latching protrusion, the second arm configured to move along the third axis. The visor assembly further includes a latching element including a track extending through a thickness of the latching element and configured to receive the latching protrusion of the second arm, the track having one or more receptacles configured to receive the latching protrusion such that the latching protrusion, when received in the one or more receptacles, prevents rotation of the first arm.

In some embodiments, the first axis intersects the first portion of the first arm and the second portion of the first arm. In some embodiments, the track includes three receptacles. In some embodiments, the visor assembly further includes a third arm coupled to the lens opposite the first arm, the third arm configured to rotate about the first axis.

In another embodiment there is a mandible shield including a panel having a top edge, a bottom edge, a left side, a right side, an inner surface, and an outer surface, the panel sized to extend across a portion of a user's face, and a first set of apertures positioned on the left side of the panel, the first set of apertures including a first aperture having a length extending along a first axis at an acute angle relative to the bottom edge of the panel, and a second aperture having a length extending along a second axis, the second axis parallel to and spaced from the first axis. The first aperture and second aperture are configured to receive a first fastener and a second fastener respectively in a plurality of positions along the length of the first and second aperture.

In some embodiments, the mandible shield further includes a second set of apertures positioned on the right side of the panel opposite the first set of apertures, the second set of apertures including a third aperture having a length extending along a third axis at an acute angle relative to the bottom edge of the panel, and a fourth aperture having a length extending along a fourth axis, the fourth axis parallel to and spaced from the third axis, and the third aperture and fourth aperture are configured to receive a third fastener and a fourth fastener respectively in a plurality of positions along the length of the third and fourth aperture. In some embodiments, the length of the third aperture and fourth aperture are about the same. In some embodiments, the first aperture and the third aperture extend along the first axis and third axis respectively at about the same acute angle relative to the bottom edge of the panel. In some embodiments, the length of the first aperture and second aperture are about the same.

In another embodiment there is an impact liner system for a helmet, the impact liner system includes an impact attenuation article comprising an aluminum honeycomb sheet having a top surface and a bottom surface, the aluminum honeycomb sheet defining a plurality of approximately hexagonally shaped cells and a cover having a top surface, a bottom surface spaced from the top surface, one or more walls coupling the top surface to the bottom surface, and one or more fasteners coupled to the top surface and configured to couple the cover to an interior of a helmet shell. The impact attenuation article is disposed within the cover between the top surface and bottom surface of the cover.

In some embodiments, the bottom surface of the cover includes one or more mounting locations each configured to receive a comfort pad. In some embodiments, the aluminum honeycomb sheet has been crushed from an initial thickness to a predetermined thickness. In some embodiments, the predetermined thickness is about 75% of the initial thickness. In some embodiments, the impact attenuation article comprises a plurality of impact attenuation articles and the cover comprises a plurality of covers. In some embodiments, the impact attenuation article includes at least four impact attenuation articles and the cover comprises four covers. In some embodiments, the one or more fasteners include one half of a hook and loop fastener.

### BRIEF DESCRIPTION OF THE DRAWINGS

The following detailed description of embodiments of the aviation helmet, will be better understood when read in conjunction with the appended drawings of an exemplary embodiment. It should be understood, however, that the invention is not limited to the precise arrangements and instrumentalities shown.

In the drawings:

FIG. 1 is a front perspective view of a helmet having accessories attached thereto in accordance with an exemplary embodiment of the present disclosure;

FIG. 2 is a rear perspective view of the helmet of FIG. 1 shown having accessories attached thereto;

FIG. 3 is a perspective view of a helmet shell of the helmet of FIG. 1;

FIG. 4 is a bottom elevational view of the helmet shell of FIG. 3 having an impact liner coupled thereto;

FIG. 5 is a side-cross sectional view of the helmet shell of FIG. 3;

FIG. 6 is a perspective view of a bare honeycomb sheet for use in the helmet of FIG. 1 cut to a desired thickness in accordance with an embodiment of the present disclosure;

FIG. 7 is a rear top perspective view of an impact liner pad of the helmet of FIG. 1;

FIG. 8 is a bottom perspective view of the impact liner pad of FIG. 7;

FIG. 9 is a front elevational view of the helmet shell of FIG. 3 having an adjustment mechanism coupled thereto;

FIG. 10 is a rear perspective view of the adjustment mechanism of FIG. 9;

FIG. 11 is a perspective view of a locking mechanism of the adjustment mechanism of FIG. 10;

FIG. 12 is a rear perspective view of the locking mechanism of FIG. 11 with the receiving member removed;

FIG. 13 is a front perspective view of the locking mechanism of FIG. 11 with the knob removed;

FIG. 14 is a side cross-sectional view of the locking mechanism of FIG. 11 with the knob removed;

FIG. 15 is a front perspective view of the helmet shell of FIG. 3 including a visor assembly;

FIG. 16 is a magnified view of a rotatable assembly of the visor assembly of FIG. 15;

FIG. 17 is a magnified view of the rotatable assembly of FIG. 16 with the latching mechanism removed;

FIG. 18 is a magnified view of the rotatable assembly of FIG. 17 with the second arm removed;

FIG. 19 is a bottom perspective view of the helmet shell of FIG. 3 with the visor and visor cover attached thereto;

FIG. 20 is a rear perspective view of the visor cover of FIG. 19;

FIG. 21 is a bottom cross-sectional elevational view of the helmet shell, visor, and visor cover of FIG. 19;

FIG. 22 is a magnified perspective view of the helmet shell of FIG. 3 having a mandible shield attached thereto;

FIG. 23 is a magnified right-side perspective view of the helmet and mandible shield of FIG. 22 with the mounting surface removed; and

FIG. 24 is a magnified left-side perspective view of the helmet and mandible shield of FIG. 22 with the mounting surface removed.

### DETAILED DESCRIPTION

There is disclosed herein a helmet and one or more accessories attached thereto. The helmet may be an aviation helmet and the one or more accessories may be accessories beneficial for aviation usage. For example, the accessories may include a nape pad adjustment mechanism configured to allow a user to precisely adjust the position of the nape pad relative to the nape of their neck thereby providing improved comfort to the user. The accessories include repositionable visor configured to allow the user to transition the visor between a usage and stowage position easily with one hand thereby allowing their remaining hand/arm to be free. The visor may also be a low-profile visor that is configured to remain close to the helmet and the user's eyes during use. The visor may be configured to be positioned between the user's face and another optical accessory such as, but not limited to, night vision goggles. The accessories also include a visor cover assembly that protects the visor and the forehead of the user from impacts and/or forces exerted on the user. The accessories also include an adjustable mandible shield that is configured to provide a greater degree of adjustability when compared to conventional mandible shields.

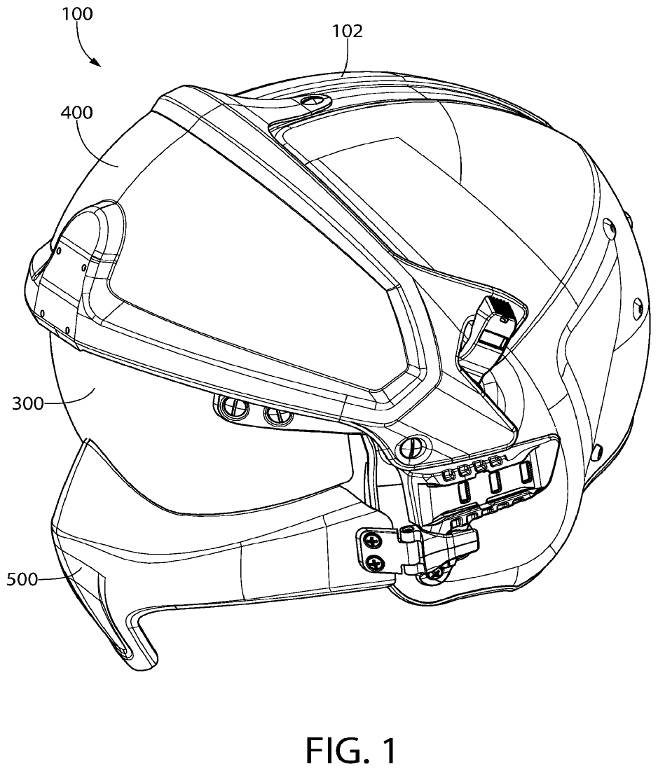

Referring to the drawings in detail, wherein like reference numerals indicate like elements throughout, there is shown in FIGS. 1-24 an aviation helmet, or helmet, generally designated 100, in accordance with an exemplary embodiment of the present invention.

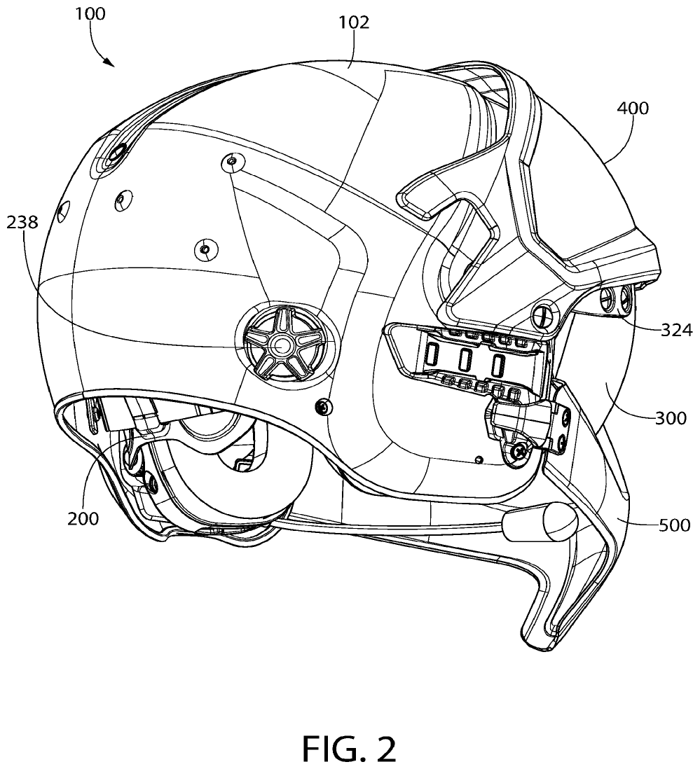

Referring to FIGS. 1-2, there is shown an embodiment of the helmet 100 having accessories attached thereto. In some embodiments, the helmet 100 is an aircraft or aviation helmet such as a helicopter helmet. In other embodiments, the helmet 100 may be configured to have one or more features applicable to an advanced combat helmet (ACH), an enhanced combat helmet (ECH), a modular integrated communications helmet (MICH), a tactical ballistic helmet (TBH), a lightweight marine helmet, police general duty helmet, a personnel armor system for ground troops (PASGT), or an aircrew helmet, such as an HGU-56/P rotary wing helmet, an HGU 55/P fixed wing helmet, an SPH-5 helmet a helicopter helmet, or any other aircrew helmet.

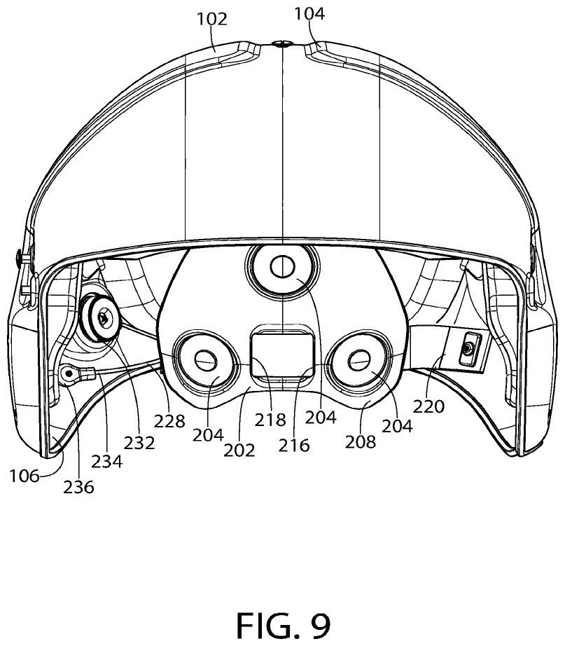

In some embodiments, the helmet 100 includes a helmet shell 102, an adjustment mechanism 200 configured to allow the user to adjust the fit of the helmet 100 to the user's head, a visor 300 for providing protection to a user's eyes, a visor cover assembly 400 for providing protection to the forehead of the user and the visor assembly 300, and a mandible shield 500 configured to provide maxillofacial protection to the user. In some embodiments, the helmet shell 102 is configured to receive the adjustment mechanism 200, the visor assembly 300, the visor cover assembly 400, and/or the mandible shield 500. In some embodiments, the visor assembly 300, visor cover assembly 400, and/or the mandible shield 500 may be coupled to the helmet shell 102 at opposing side surfaces of the helmet shell 102. In some embodiments, the visor assembly 300, visor cover assembly 400, and/or the mandible shield 500 may extend from a first side surface of the helmet shell 102 to or outwardly from a front of the helmet shell 102 and to a second side surface that is opposite the front side surface. In this manner, the visor assembly 300, visor cover assembly 400, and/or the mandible shield 500 may protect portions of the user's face when the helmet 100 is worn by the user. In some embodiments, the adjustment mechanism 200 may be coupled to a rear of the helmet shell 102. In some embodiments, the adjustment mechanism 200 may include a knob 238 operatively coupled to the adjustment mechanism 200 and configured to allow a user to rotate the knob 238 to adjust the fit of the helmet 100 to the user's head, as discussed in more detail with reference to FIGS. 9-14.

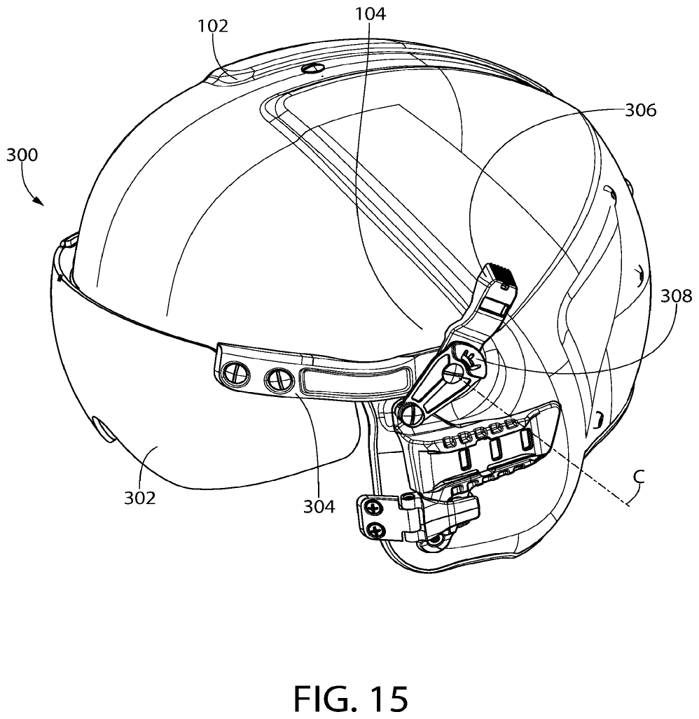

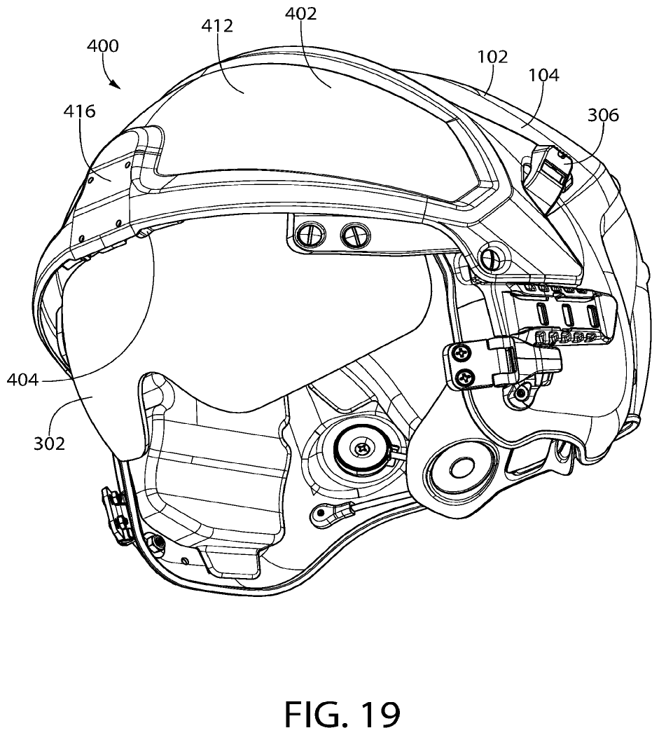

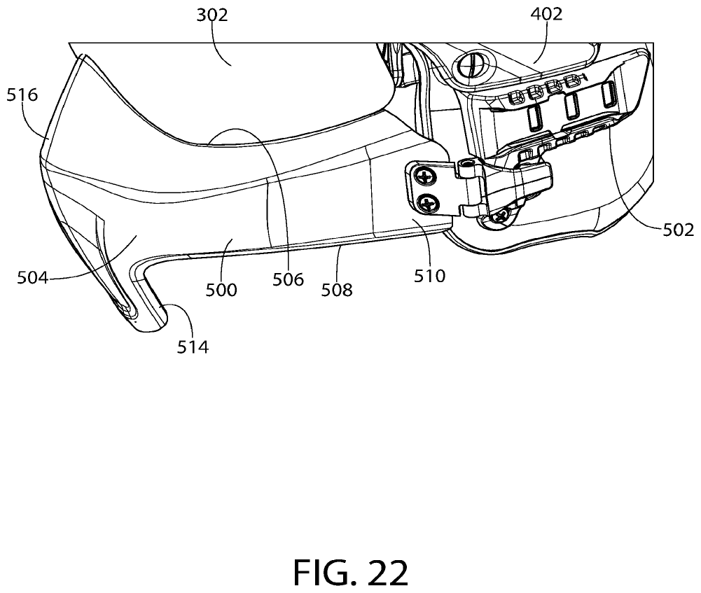

In some embodiments, the visor assembly 300 may be configured to pivot relative to the helmet shell 102 between two or more use positions and a stowage position, as discussed in more detail with reference to FIGS. 15-18. In some embodiments, the visor cover assembly 400 may be configured to cover the visor assembly 300 when the visor is in the stowage position and provide protection from impacts, as discussed in more detail with reference to FIGS. 19-21. In some embodiments, the mandible shield 500 may be configured to be adjustable relative to the helmet shell 102 to allow a user to adjust the position of the mandible shield 500 relative to the user's face when the user is wearing the helmet 100, as discussed in more detail with reference to FIGS. 22-24.

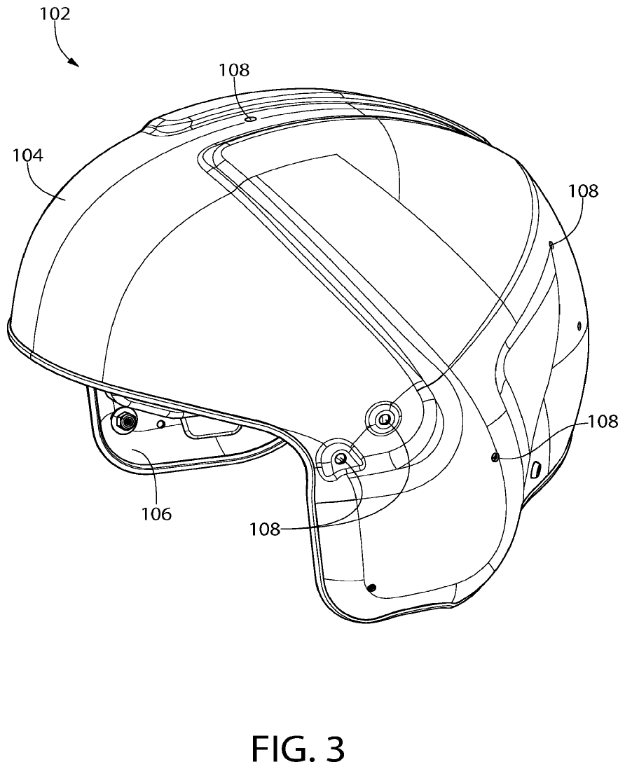

Referring to FIG. 3, the helmet shell 102 may have an outer surface 104 and an inner surface 106 disposed opposite the outer surface 104. In some embodiments, the helmet shell 102 may be configured to cover the cranium, ears, and back of the user's head. In some embodiments, the helmet shell 102 may be shaped such that the user's eyes, nose and mouth are not covered by the helmet shell 102. In some embodiments, the helmet shell 102 may be comprised of a ballistic material. In some embodiments, the helmet shell 102 may include one or more mounting locations 108 disposed along the helmet shell for coupling accessories to the helmet shell 102. In some embodiments, at least one of the one or more mounting locations extend from the outer surface 104 to the inner surface 106 of the helmet shell 102. In some embodiments, at least one of the one or more mounting locations 108 do not extend through the thickness of the helmet shell 102. In some embodiments, the one or more mounting locations 108 may be sized to receive fasteners (e.g., screws, bolts) for selectively coupling one or more accessories (e.g., adjustment mechanism 200, visor assembly 300, visor cover assembly 400, mandible shield 500) to the helmet shell 102. In some embodiments, the one or more accessories may be coupled and/or decoupled from the helmet shell 102. For example, fasteners which extend through the mounting locations 108 may be removed in order to decouple one or more accessories from the helmet shell 102.

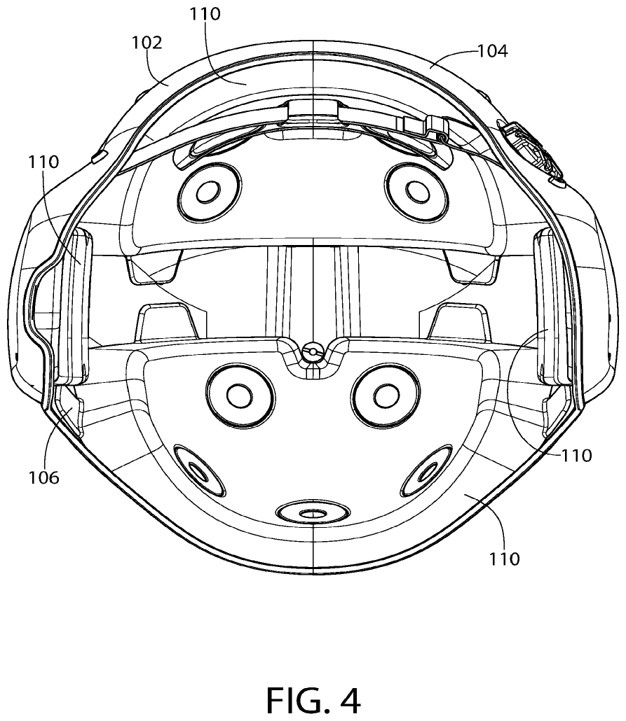

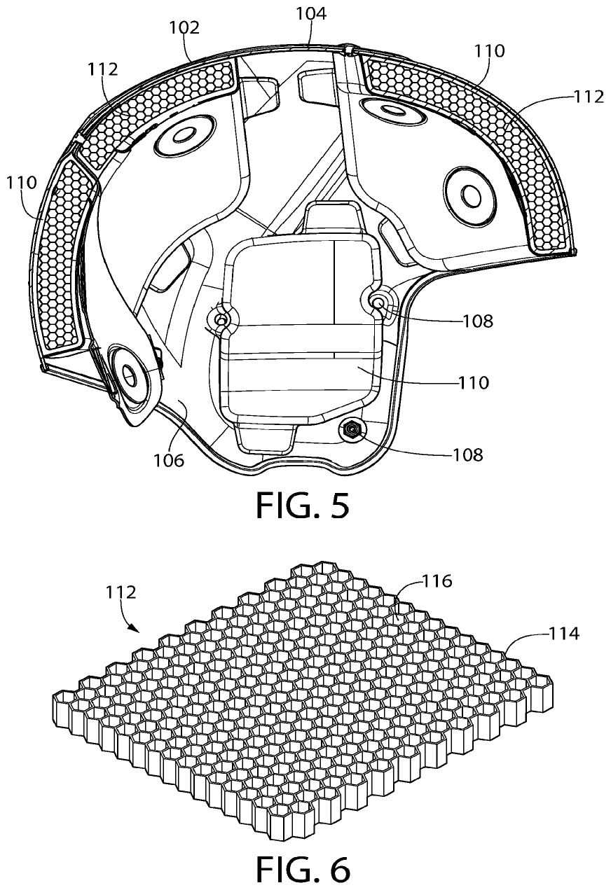

Referring to FIGS. 4-5 the helmet 100 may include an impact liner system including one or more covers 110, also referred to as liners, and one or more impact attenuation articles 112 configured to improve the impact attenuation of the helmet 100. In some embodiments, the impact liner system may be configured to be positioned along the inner surface 106 of the helmet shell 102. More particularly, the impact liner system, in some embodiments, is configured to be positioned between the helmet shell 102 and the user's head during use and provide impact protection to the user. In some embodiments, the impact liner system may be removably attached to the helmet shell 102. For example, covers 110 may be configured to be removably attached to the inner surface 106 of the helmet shell 102. In some embodiments, one or more of the covers 110 is coupled to the inner surface 106 of the helmet shell 102 via fasteners extending through one or more of the mounting locations 108 on the helmet shell 102. In some embodiments, one or more of the covers 110 is coupled to the inner surface 106 of the helmet shell 102 via fastening means (e.g., hook and loop fastener, adhesive strips) disposed on the inner surface 106 of the helmet shell 102.

In some embodiments, each cover 110 is configured to enclose a corresponding impact attenuation article 112. In some embodiments, each cover 110 may define an interior chamber for receiving a corresponding impact attenuation article. In some embodiments, the walls of the cover 110 which define the interior chamber may be substantially thinner than the corresponding impact attenuation article 112. The covers 110 and corresponding impact attenuation articles 112 may define any shape to provide the desired impact attenuation. For example, in some embodiments, cover 110 and the impact attenuation article 112 may define a shape to fit snugly within an opening, hole, or recess in the helmet shell 102, or any other conventional helmet shell. In some embodiments, the covers 110 and impact attenuation articles 112 may be sized and shaped to cover various areas of the inner surface 106 of the helmet shell 102. For example, in the embodiment shown in FIGS. 4-5 the covers 110 cover portions of the left side, right side, top, front and rear of the inner surface 106 of the helmet shell 102. In some embodiments, there may be gaps between one or more of the covers 110. In other embodiments, there may be a single cover 110, and impact attenuation article disposed therein, that is sized and shaped to cover all or substantially all of the inner surface 106 of the helmet shell 102. For example, the covers 110, illustrated in FIGS. 4-5 may be integrally formed with one another such that there are no gaps between adjacent covers 110.

Referring to FIG. 6, In some embodiments, each impact attenuation article 112 may comprise an aluminum honeycomb sheet 102. In some embodiments, sheet 102 is a pre-crushed aluminum honeycomb sheet. A pre-crushed aluminum honeycomb sheet as used herein refers to an aluminum honeycomb material that has been treated with a controlled impact (e.g., pre-crushed), or otherwise has had a portion of the aluminum honeycomb sheet reduced in thickness from an original height to a predetermined height such as by cutting or sanding. In some embodiments, sheet 102 is comprised of a lightweight material that may meet standardized impact requirements while potentially maintaining performance through multiple impact test hits. The combination of material, geometry and use may allow sheet 102 to be used for impact attenuation in helmets. In some embodiments, sheet 102 defines a plurality of cells 116 having generally the same shape (e.g., approximately hexagonally shaped cells). In some embodiments, the sheet 102 may be pre-crushed to a predetermined thickness of about 0.1%, 0.5%, 1%, 2%, 5%, 7%, 10%, 15%, 20%, 25%, 30%, 35%, 40%, 45%, 50%, 55%, 60%, 70%, 75%, 80%, 85%, or 95% of the initial thickness.

Although sheet 102 may ordinarily comprise or consist essentially of aluminum, in some embodiments, sheet 102 may comprise other materials, plastics, alloys or one or more composites. Also, although in some embodiments, the pre-crushed aluminum honeycomb sheet 102 includes generally hollow cells 116 each having a generally hexagonally shaped cross section with a generally constant size, the cells 116 may, in some embodiments, have a different cross-sectional shape such as triangular, circular, rectangular, pentagonal, hexagonal, or other shapes such as a polygonal shape. Preferably, cells 116 are approximately hexagonally shaped. In some embodiments, by using an impact attenuation article comprising an aluminum honeycomb sheet 102, the overall thickness of the impact liner system may be reduced, as compared to traditional impact liner systems, while maintaining or potentially improving the impact attenuation capabilities of the helmet 100.

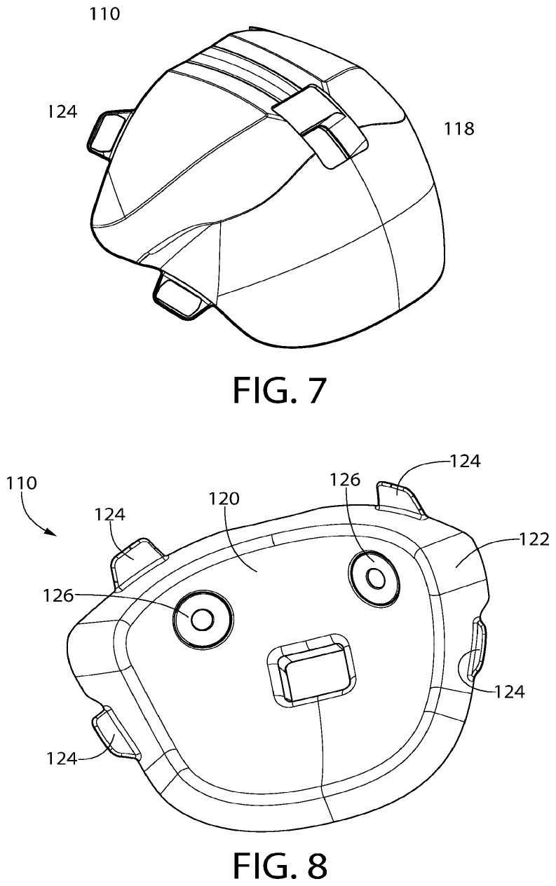

Referring to FIGS. 7-8, in some embodiments, the cover 110 may include a top surface 118 and a bottom surface 120 spaced from the top surface 118. In some embodiments, the cover 110 may include a sidewall 122 extending around a periphery of the top surface 118 and coupling the top surface 118 to the bottom surface 120. In some embodiments, the top surface 118, bottom surface 120, and sidewall 122 may define the interior chamber where the impact attenuation article 112 is disposed. In some embodiments, the top surface 118 of the cover 110 may include one or more fasteners 124 configured to couple the cover 110 to the inner surface 106 of the helmet shell 102. In some embodiments, substantially all of the top surface 118 of the cover 110 includes a fastener configured to couple the cover 110 to the inner surface 106 of the helmet shell 102. For example, substantially all of the top surface 118 of the cover 110 may include a layer of one half of a hook and loop fastener, preferably the loop fastener, for removably coupling to the other half of the hook and loop fastener which is coupled to the inner surface 106 of helmet 102.

In some embodiments, the layer of fasteners (e.g., loop fasteners) coupled to the top surface 118 may be configured to reduce prevent or at least reduce the impact attenuation article 112 disposed within the cover 110 from scratching or rubbing against the inner surface 106 of the helmet shell 102. In some embodiments, the layer of fasteners may be sandwiched between the impact attenuation article 112 and the inner surface 106 of the helmet shell 102 to mitigate the sound generated when the aluminum honeycomb sheet 114 deforms or crinkles.

In some embodiments, the bottom surface 120 of the cover 110 includes one or more mounting locations 126 for receiving one or more comfort pads (not shown). In some embodiments, the mounting locations 126 may include a fastener configured to couple to a corresponding fastener of the one or more comfort pads. For example, the mounting locations 126 may include one half of a hook and loop fastener and the one or more comfort pads may include the other half of the hook and loop fastener. In some embodiments, each cover 110 included in the impact liner system (e.g., covers 110 shown in FIG. 4) includes at least one mounting location 126 for receiving a comfort pad. In other embodiments, not all covers 110 included in the impact liner system include a mounting location 126 for receiving a comfort pad. In some embodiments, the covers 110 include one, two, three, four, five, or more than five mounting locations 126 for receiving a comfort pad.

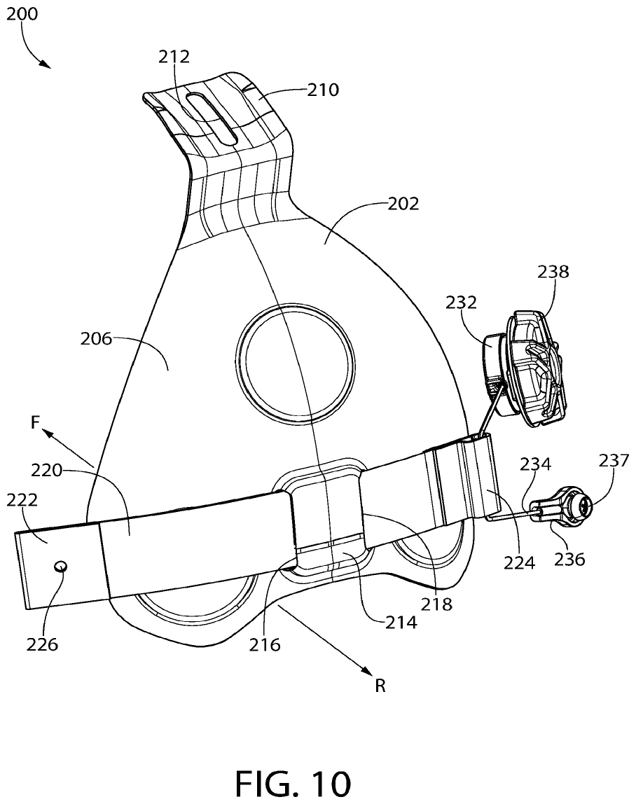

Referring to FIGS. 9-10, in some embodiments, the adjustment mechanism 200 may be coupled to the inner surface 106 of the helmet shell 102. The adjustment mechanism 200 may include a base 202 configured to be positioned proximal the nape of a user's neck when the helmet 100 is worn by the user. For example, the base 202 may be the base plate of a nape pad that is configured to be positioned proximal the nape of the user's neck. In some embodiments, the base 202 is configured to receive at least one compressible comfort pad (not shown) such that the comfort pad contacts the nape of the user's neck when the helmet 100 is worn by the user. The base 202 with the one or more compressible comfort pads coupled thereto may be referred to as a nape pad. The base 202 may include one or more mounting surfaces 204 configured to receive a compressible comfort pad. In some embodiments, the one or more mounting surfaces 204 may include a hook and loop fastener.

In some embodiments, the base 202 may include one, two, three, four or more than four mounting surfaces 204. In some embodiments, the base 202 is comprised of a rigid material. In some embodiments, the base 202 includes an outer surface 206 and an inner surface 208 disposed opposite the outer surface 206. In some embodiments, the outer surface 206 is generally convex and the inner surface 208 is generally concave. In some embodiments, the base 202 may include an arm 210 extending from the top of the base 202 and configured to couple the base 202 to the helmet shell 102. For example, arm 210 may define an opening 212 configured to receive a fastener extending through one of the mounting locations 108 of helmet shell 102 to removably couple the base 202 to the helmet shell 102. In some embodiments, the arm 210 couples to a fastener extending through a mounting location 108 proximal a top portion of the helmet shell 102.

In some embodiments, the base 202 may include a protrusion 214 extending outwardly from the outer surface 206. The protrusion 214 may define a first aperture 216 disposed on a first side of the aperture 216 and a second aperture 218 disposed on a second side of the aperture 216 opposite the first side. In some embodiments, the protrusion 214 may be sized to receive a strap 220. The strap 220 may be sized to extend through the first aperture 216 and the second aperture 218. In some embodiments, the strap 220 is a comprised of a flexible material. In other embodiments, the strap is comprised of a rigid material. In some embodiments, the strap 220 includes a first end 222 configured to couple to the helmet shell 102 and a second end 224 opposite the first end 222. In some embodiments, the first end 222 may define an opening 226 configured to receive a fastener to fixedly couple the first end 222 of the strap 220 to the helmet shell 102. In some embodiments, the first end 222 of the strap 220 may be fixed relative to the helmet shell 102. The second end 224 of strap 220 may be looped around a cable 228 coupled to the helmet shell 102. The second end 224 of the strap 220 may be looped around the cable 228 such that displacement of the cable 228 causes the second end 224 of the strap 220 to be displaced relative to the helmet shell 102.

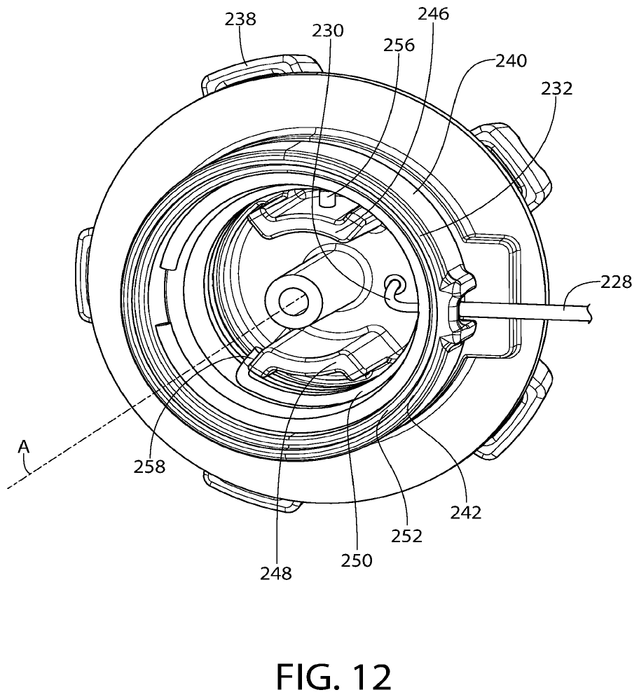

In some embodiments, the cable 228 includes a first end 230, as shown in FIG. 12, extending into a housing 232 that is configured to be coupled to the helmet shell 102. In some embodiments, the cable 228 includes a second end 234 that is coupled to the helmet shell 102 such that the second end 234 of cable 228 is fixed relative to the helmet shell 102. In some embodiments, the second end 234 of cable 228 may extend into a cable housing 236 which is configured to receive the second end 234 of the cable 228 and couple to the inner surface 106 of helmet shell 102. The second end 234 of the cable 228 may include a loop that is, although not shown in FIGS. 9-10, configured to be positioned within the cable housing 236 such that a fastener 237 may extend through the loop in the second end 234 of the cable 228 to fixedly secure the second end 234 of the cable 228 and the cable housing 236 to the inner surface 106 of the helmet shell 102.

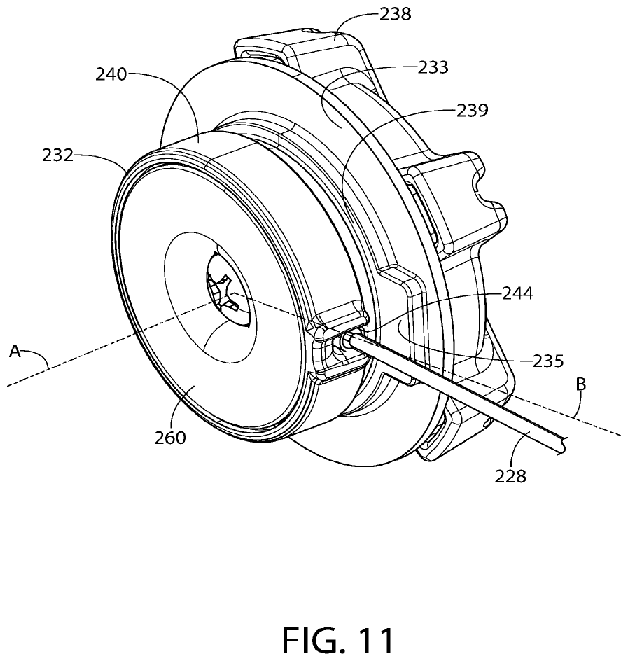

Referring to FIGS. 11-12, in some embodiments, the housing 232 may be configured to receive knob 238 such that the knob 238 is rotatable about a central axis A. The housing 232 may include a sidewall 240 extending circumferentially around the central axis A. In some embodiments, the sidewall 240 defines an opening 242 extending along the central axis A. The sidewall 240 may define an aperture 244 extending through the sidewall 240 along a transverse axis B that is perpendicular to the central axis A. In some embodiments, the housing 232 is configured to remain fixed relative to the helmet shell 102 when the knob 238 is rotated. For example, the housing 232 may not rotate in response to the knob 238 being rotated (e.g., manually rotated by a user) about the central axis A. In some embodiments, the knob 238 may include a first protrusion 246 and a second protrusion 248 extending into the opening 242. In some embodiments, the first protrusion 246 and the second protrusion 248 have generally the same shape. In some embodiments, the first protrusion 246 and the second protrusion 248 are disposed opposite one another relative to the central axis A. In some embodiments, the first protrusion 246 and the second protrusion 248 are integrally formed with the knob 238 such that rotation of the knob 238 about the central axis A causes the first protrusion 246 and the second protrusion 248 to rotate about the central axis A.

In some embodiments, the housing 232 may be configured to couple to the helmet shell 102, as shown in FIGS. 2 and 9. In some embodiments, the housing 232 may include a lip 233 extending circumferentially around the central axis A and outwardly from sidewall 240. In some embodiments, the lip 233 has a diameter which is greater than the diameter of sidewall 240. In some embodiments, the sidewall 240 is sized to extend through an opening in helmet shell 102 (not shown). In some embodiments, the opening in the helmet shell 102 for receiving the sidewall 240 of housing 232 has a diameter which is less than the diameter of lip 233. In some embodiments, the lip 233 includes a tooth 235 configured to engage with a corresponding aperture in the opening in the helmet shell 102 such that the housing 232 is prevented from rotating relative to the helmet shell 102 when the tooth 235 is engaged with the aperture. In some embodiments, the lip 233 is configured to couple to the outer surface 104 of the helmet shell. In some embodiments, the housing 232 includes a retention ring 239 configured to contact the inner surface 106 of the helmet shell when the housing 232 is coupled to the helmet shell 102.

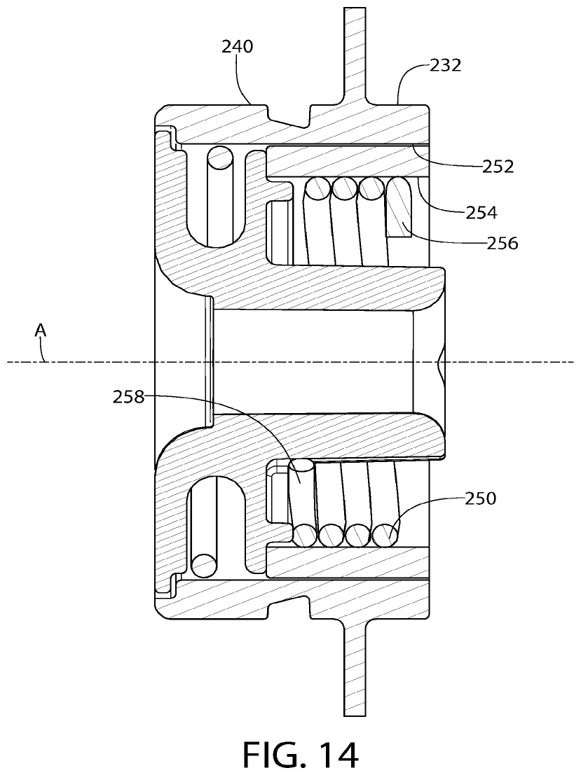

In some embodiments, the first protrusion 246 and the second protrusion 248 may engage a locking element 250 disposed within housing 232. In some embodiments, the locking element 250 may be engaged with an interior surface 252 of the sidewall 240 of the housing 232. In some embodiments, the locking element 250 is engaged with a collar 254 (shown in FIG. 14) coupled to the inner surface 252 of the sidewall 240 of housing 232. In some embodiments, the locking element 250 may extend circumferentially around at least a portion of the interior surface 252 of the sidewall 240 generally about central axis A. In some embodiments, the locking element 250 may comprise a biasing element (e.g., a spring) configured to engage with the interior surface 252 of the sidewall 240 and configured to disengage from the interior surface 252 of the sidewall 240 in response to manual rotation of the knob 238 by a user. In some embodiments, the locking element 250 may include a first terminus 256 and a second terminus 258 extending towards central axis A, and configured to engage the first protrusion 246 and second protrusion 248 of the knob 238. For example, the first terminus 256 of the locking element 250 may be engaged with the first protrusion 246 of the knob 238 such that rotation of the knob 238 causes the first terminus 256 of the locking element 250 to rotate. Similarly, the second terminus 258 of the locking element 250 may be engaged with the second protrusion 248 such that rotation of the knob 238 causes the second terminus 258 of the locking element 250 to rotate.

In some embodiments, rotation of the first terminus 256 and the second terminus 258 of the locking element 250 may cause the locking element 250 to constrict, or compress, such that the diameter of the locking element 250 is reduced from a first diameter to a second diameter causing the locking element 250 to disengage with the interior surface 252 of the sidewall 240 of the housing 232. In some embodiments, when the locking element 250 is engaged with the housing 232, the knob 238 may be prevented from being unintentionally rotated. For example, when the locking element 250 is engaged with the sidewall 240 of the housing, the locking element 250 may resist rotation of the knob 238 that, when the user manually rotates the knob 238, may be overcome allowing the knob to be rotated. In some embodiments, the first terminus 256 and second terminus 258 may be disposed on opposite ends of the locking element 250.

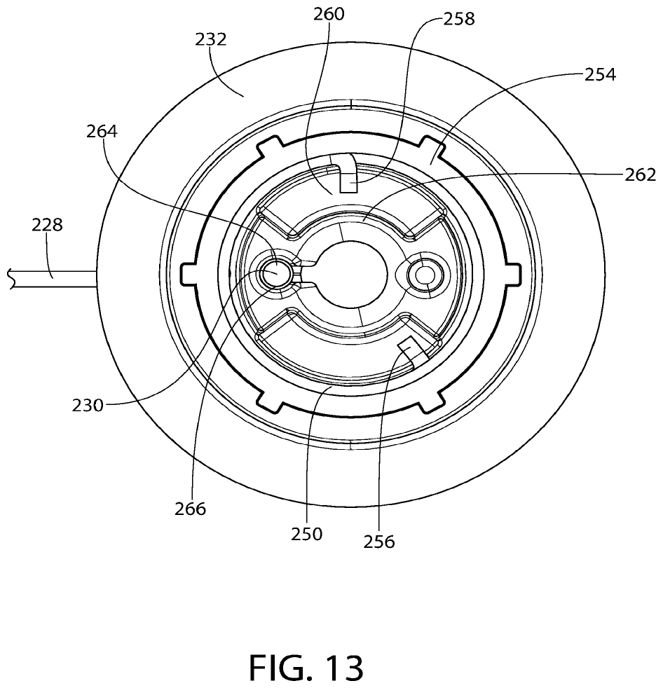

Referring to FIGS. 11 and 13, the adjustment mechanism 200 may include a receiving member 260 rotatably coupled to housing 232. In some embodiments, the receiving member 260 may be rotatable about central axis A. In some embodiments, the receiving member 260 may include a protrusion 262 extending toward the knob 238 along central axis A. In some embodiments, the protrusion 262 may define an aperture 264 extending along an axis (not shown) which is generally parallel to and spaced from the central axis A. For example, the axis along which the aperture 264 extends may extend relative to the central axis A such that rotation of the receiving member about central axis A causes the aperture 264 to rotate about the central axis A. In some embodiments, protrusion 262 is sized and shaped to extend within opening 242 along central axis A such that the protrusion 262 is interior to the locking element 250. In some embodiments, the protrusion 262 is configured to engage the first protrusion 246 and/or the second protrusion 248 of knob 238 such that rotation of the knob 238 about central axis A causes the receiving member 260 to rotate about the central axis A.

In some embodiments, the first end 230 of cable 228 may be fixedly coupled to the receiving member 260 such that the first end 230 of cable 228 rotates about the central axis A when the receiving member 260 rotates about the central axis. In some embodiments, the first end 230 of the cable 228 may extend through aperture 244 into the opening 242 defined by the sidewall 240 and through the aperture 264 defined by the protrusion 262 of the receiving member. In some embodiments, the first end 230 of the cable 228 may be disposed within aperture 264 and fixedly coupled to the receiving member by a sleeve 266. In some embodiments, the sleeve 266 may be a deformable material configured to be crimped to the first end 230 of cable 228. In some embodiments, the first end 230 of the cable 228 being fixedly disposed within aperture 244 causes the first end 230 of the cable 228 to be rotated about the central axis A in response to the knob 238 being rotated about the central axis A.

Referring to FIGS. 10 and 11, in some embodiments, rotation of the knob 238 about the central axis A may cause the base 202 to be adjusted relative to the helmet shell 102. In some embodiments, rotation of the knob 238 in a first direction may cause a portion of cable 228 to wind within housing 232 about the central axis A. For example, rotation of the knob in a clockwise direction may cause a portion of cable 228 to wind within housing 232 such that a portion of the cable 228 which was previously disposed external to the housing 232 is disposed within the housing 232. In some embodiments, rotation of the knob 238 in a second direction opposite the first direction may cause a portion of cable 228 to unwind within housing 232 about the central axis A. For example, rotation of the knob 238 in a counterclockwise direction may cause a portion of cable 228 to unwind within the housing 232 such that a portion of cable 228 disposed within housing 232 is disposed external to the housing 232. The cable 228 winding or unwinding within housing 232 may in turn cause the second end 224 of strap 220 to be displaced relative to the helmet shell 102.

In some embodiments, displacement of the second end 224 of strap 220 may cause the base 202 to be displaced relative to the helmet shell 102. In some embodiments, the base 202 may be displaced relative to the helmet shell 102 in a forward direction F or a rearward direction R such that the base 202 is moved towards or away from the nape of the user's neck when the helmet 100 is worn by the user. In some embodiments, the arm 210 coupling the base 202 to helmet shell 102 may remain fixed relative to the helmet shell 102 during displacement of the base 202. In some embodiments, the portion of the base 202 where the arm 210 is coupled to the base 202 is configured to elastically deform in response to displacement of the base 202 relative to the housing 232 and/or helmet shell 102. For example, if the knob 238 is rotated by a user about axis A such that the cable 228 is wound within housing 232 (e.g., a portion of the cable is moved into the housing 232), the base 202 may be displaced relative to the helmet shell 102 such that the arm 210 is elastically deformed allowing the base 202 to be moved closer to the user's neck and/or head. In this manner, the arm 210 may act as a biasing element that exerts a force on the base 202 to cause it to move back to a position in which the arm is not elastically deformed. In some embodiments, the cable 228 and locking element 250 may be configured to resist displacement of the base 202 in the rearward direction when the arm 210 is elastically deformed. For example, the engagement of the locking element 250 with the interior surface 252 of sidewall 240 may prevent the cable 228 from unwinding within the housing 232 in response to the force of the base 202 on the cable 228 caused by the elastic deformation. Similarly, rotating the knob 238 in the opposite direction may unwind an amount of the cable 228 from within housing 232 allowing the base 202 to move rearward towards a neutral position where the arm 210 is not elastically deformed.

In some embodiments, the winding and unwinding of cable 228 within housing 232 to move the base 202 toward or away from the user's neck may provide the user a more precise amount of control over the placement of the base 202 when compared to conventional adjustment mechanisms. Conventional adjustment mechanisms often employ a rack and pawl configuration which has a number of predetermined finite stops spaced along the rack. The adjustment mechanism 200 of the present disclosure may be configured to have no finite stops and may automatically lock in place following a manual rotation of knob 238 by the user. For example, locking element 250 may automatically engage with the interior surface 252 of the sidewall 240 when the knob 238 is not being manually rotated. Additionally, by providing a cable 228 as opposed to other conventional methods, the adjustment mechanism may be generally silent when used to adjust the position of the base 202 relative to the user's neck and/or head.

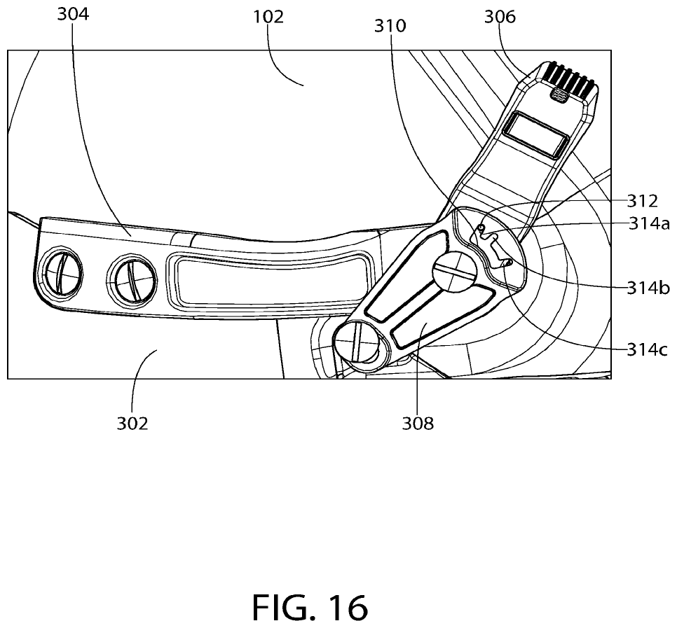

Referring to FIGS. 15-16, in some embodiments, the helmet 100 may include a visor assembly 300. In some embodiments, the visor assembly 300 may include a lens 302 sized to extend across at least a portion of a user's face, a first arm 304 coupled to the lens 302 and rotatable about an axis C. In some embodiments, rotation of the first arm 304 about axis C causes the lens 302 to rotate about axis C. In some embodiments, the visor assembly 300 may include a second arm 306 coupled to the first arm 304 and rotatable about axis C. In some embodiments, the first arm 304 may be operatively engaged with the second arm 306 such that rotation of the second arm about axis C causes the first arm 304 and lens 302 to rotate about axis C. In some embodiments, the visor assembly 300 may include a latching element 308 engaged with the second arm 306 and configured to releasably lock the lens 302 in a plurality of positions. In some embodiments, the latching element 308 includes a track 310 extending through a thickness of the latching element 308. In some embodiments, the track 310 may be sized to receive a latching protrusion 312 coupled to the second arm 306. In some embodiments, the latching protrusion 312 may move along track 310 when the second arm 306 is rotated above axis C. In some embodiments, the first arm 304 and second arm 306 may be sandwiched between the outer surface 104 of helmet shell 102 and the latching element 308.

In some embodiments the track 310 may include one or more receptacles 314 configured to releasably retain the second arm 306 in one or more predetermined locations. For example, the track 310 may include receptacles 314a, 314b, and 314c disposed at different positions along track 310. In some embodiments, the receptacles 314a, 314b, and 314c are sized to receive the latching protrusion 312 such that the second arm 306 is prevented from being rotated about axis C while the latching protrusion 312 is received in any one of receptacles 314a, 314b, and 314c. In some embodiments, when the latching protrusion 312 is received in any one of receptacles 314a, 314b, and 314c, the first arm 304 may be prevented from rotating about axis C. In the embodiment shown in FIGS. 15-16, the track 310 includes three receptacles 314a, 314b, and 314c, however, in some embodiments, the track 310 may include one receptacle, two receptacles, three receptacles, four receptacles, five receptacles, or more than five receptacles.

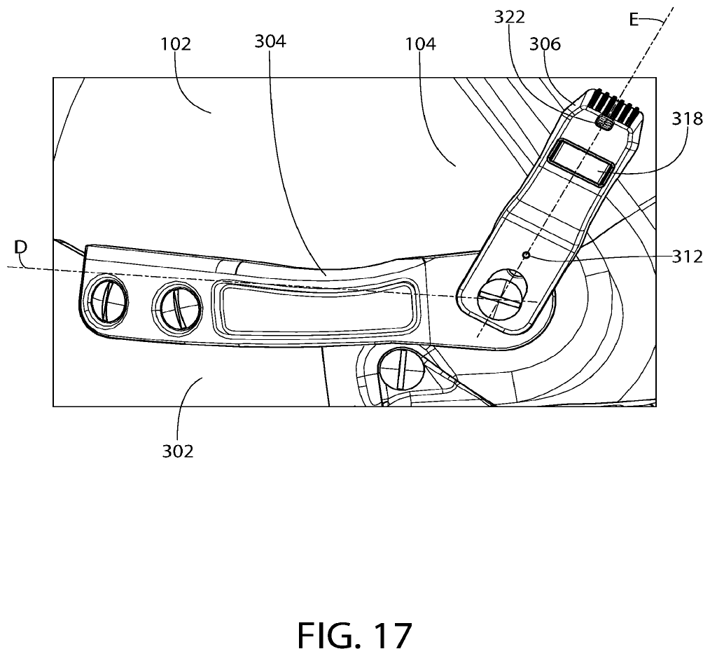

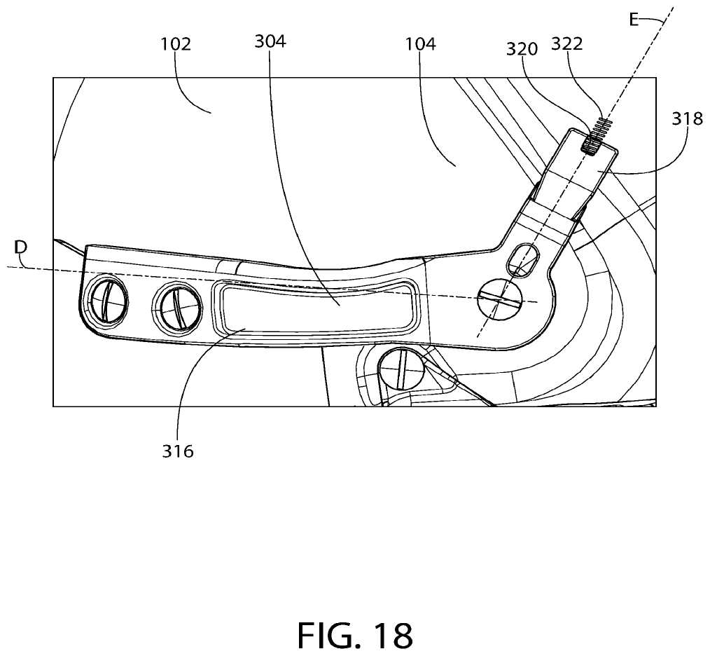

Referring to FIGS. 18, the first arm 304 may include a first portion 316 extending generally along an axis D and a second portion 318 extending from an end of the first portion 316 generally along an axis E. In some embodiments, axis E may be at an obtuse angle relative to axis D such that the first arm 304 forms a bent shape. In some embodiments, the second portion 318 may include a receiving area 320 for receiving a biasing element (e.g., biasing element 322 shown in FIG. 17) of the second arm 306. In some embodiments, axis C intersects the first portion 316 and the second portion 318.

Referring to FIG. 17, the second arm 306 may include a biasing element 322 coupled to the second arm 306 configured to be received within receiving area 320 of the first arm 304. In some embodiments, the second arm 306 may be configured to translate along axis E towards or away form the axis C. In some embodiments, the biasing element 322 biases the second arm 306 away from axis C. In some embodiments, the user may apply a force to the second arm 306 causing the second arm 306 to translate along axis E such that the second arm 306 is moved toward the axis C. In some embodiments, translation of the second arm 306 toward axis C may cause the latching protrusion 312 to disengage with one of receptacles 314a-314c of the latching element 308 such that the latching protrusion 312 is received within track 310 and is rotatable about axis C.

Referring to FIGS. 2 and 15, in some embodiments, the visor assembly 300 may include a third arm 324 coupled to lens 302 opposite the first arm 304. In some embodiments, the third arm 324 may be rotatable about axis C. In some embodiments, rotation of the first arm 304 about axis C causes the third arm 324 to rotate about axis C such that the lens 302 rotates about axis C. In some embodiments, a fourth arm (not shown) similar to the second arm 306 and a second latching member (not shown) similar to latching member 308 may be coupled to the third arm 324. In other embodiments, the third arm 324 may not include the fourth arm and second latching member. In some embodiments, by providing a visor assembly 300 having the first arm 304, second arm 306 and latching member 308 as discussed above, the lens 302 of the visor assembly 300 may remain close to the user's face which may in turn allow the thickness of the helmet shell 102 under the lens 302 to be reduced. This reduction of thickness of the helmet shell 102 may be compensated by providing a visor cover assembly having one or more impact attenuation pads.

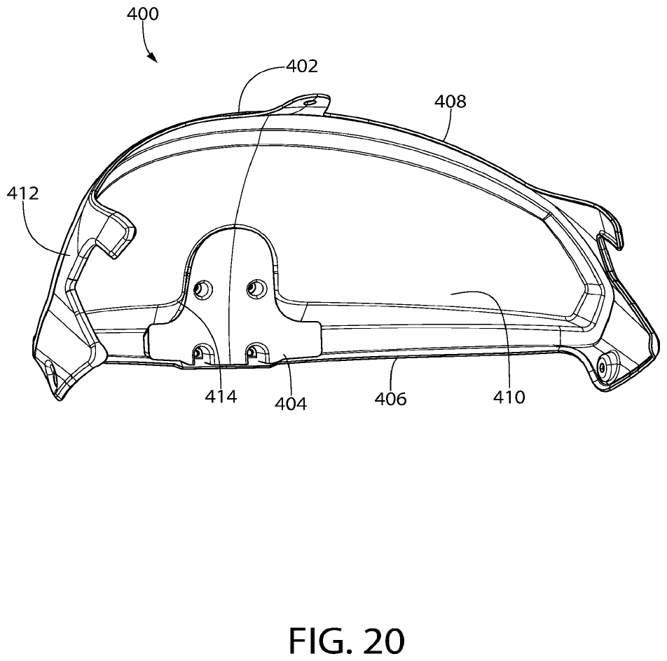

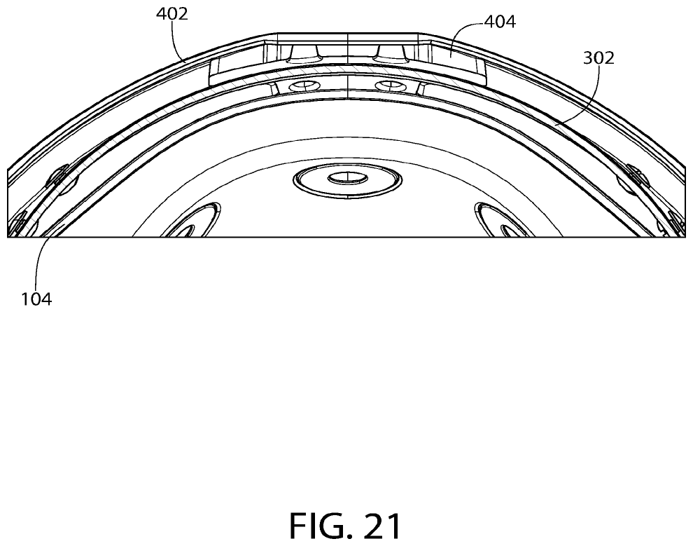

Referring to FIGS. 19-20, in some embodiments, the helmet 100 may include a visor cover assembly 400 including a cover 402 and an impact attenuation pad 404 coupled to the cover 402. In some embodiments, the cover 402 may be coupled to the outer surface 104 of helmet shell 102. In some embodiments, the cover 402 may be spaced from the outer surface 104 of the helmet shell 102 such that the lens 302 may be positioned between the cover 402 and the outer surface 104 of the helmet shell 102 when the visor is in a stowage position, as shown in FIG. 21. In some embodiments, the cover 402 may be sized to cover at least a portion of the latching element 308, the first arm 304, and the second arm 306 of the visor assembly 300. In some embodiments, the cover 402 may have a bottom edge 406, a top edge 408 opposite the bottom edge 406, an inner surface 410 and an outer surface 412 opposite the inner surface 410. In some embodiments, the inner surface 410 may include a recessed receiving area 414 proximal the bottom edge 406. In some embodiments, the impact attenuation pad 404 may be coupled to the inner surface 410 and positioned within recessed receiving area 414. In some embodiments, by providing the impact attenuation pad 404 within the recessed receiving area 414, the impact attenuation performance of the helmet 100 may be improved while minimizing the distance from the center of the helmet 100 to the impact attenuation pad 404 thereby keeping the center of gravity of the helmet 100 close to the head of a user wearing the helmet 100.

In some embodiments, the recessed receiving area 414 and impact attenuation pad 404 are centrally located on the inner surface 410 of the cover 402 proximal the bottom edge 406. In other embodiments, the recessed receiving area 414 and impact attenuation pad 404 are spaced from the bottom edge 406. In some embodiments, the outer surface 412 may include a mounting area 416 configured to receive at least one of a plurality of accessories (e.g., night vision goggles, binoculars). In some embodiments, the mounting area 416 extends outwardly from the outer surface 412. In some embodiments, the mounting area 416 may be disposed proximal the recessed receiving area 414 such that the recessed receiving area 414 is recessed within the mounting area 416. In some embodiments, the impact attenuation pad 404 may have a thickness which is less than or equal to the depth of the recessed receiving area 414. In other embodiments, the impact attenuation pad 404 has a thickness which is slightly larger than the depth of the recessed receiving area 414. In some embodiments, the visor cover assembly 400 may include at least one other impact attenuation pad coupled to the inner surface 410 at a location different from impact attenuation pad 404. In other embodiments, the impact attenuation pad 404 may be sized to extend along substantially all of the inner surface 410 of the cover 402. In some embodiments, the impact attenuation pad 404 is coupled to the inner surface 410 via fasteners, adhesives, or a combination thereof.

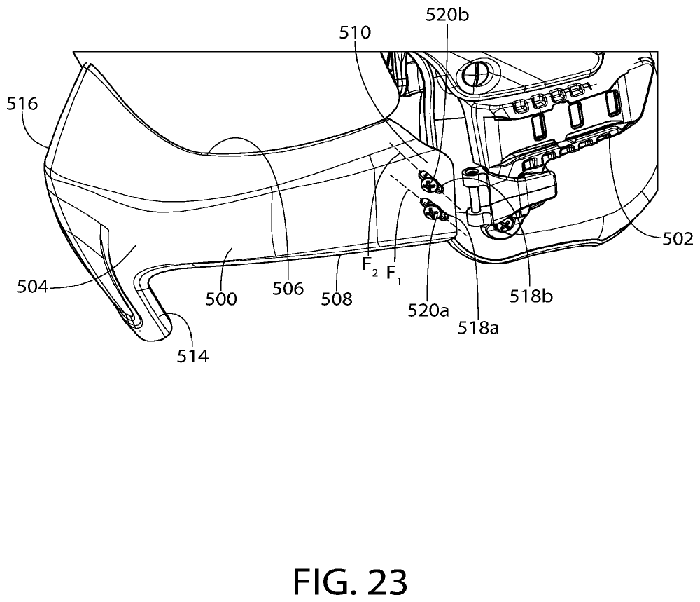

Referring to FIGS. 22-23, the helmet 100 may be configured to receive a mandible shield 500. In some embodiments, there may be one or more mounting rails 502 coupled to the outer surface 104 of helmet shell 102 configured to couple the mandible shield 500 to the helmet shell 102. In some embodiments, the mandible shield may comprise a panel 504 having a top edge 506, a bottom edge 508, a left side 510, a right side 512, an inner surface 514, and an outer surface 516. In some embodiments, the panel 504 is sized to extend across a portion of the user's face. In some embodiments, the panel 504 is sized to extend across the user's mouth nose and chin. In some embodiments, the mandible shield 500 may include a first pair of apertures including a first aperture 518a and second aperture 518b positioned on the left side 510 of panel 504. In some embodiments, the first pair of apertures 518a and 518b may extend through the thickness of panel 504 from the outer surface 516 to the inner surface 514.

In some embodiments, the first aperture 518a may extend along an axis F₁ and the second aperture 518b may extend along an axis F₂. In some embodiments, the axes F₁ and F₂ may be generally parallel and spaced from one another. In other embodiments, the axes F₁ and F₂ may not be generally parallel. In some embodiments, the axes F₁ and F₂ may each be at an acute angle relative to the bottom edge 508 of the panel. Each aperture 518a and 518b may have a length extending along the corresponding axes F₁ and F₂. In some embodiments, each aperture 518a and 518b have generally the same length. In other embodiments, aperture 518a may have a length which is different than the length of aperture 518b. In some embodiments, the first aperture 518a and second aperture 518b are each configured to receive a corresponding fastener 520a and 520b for adjustably coupling the mandible shield 500 to the mounting rail 502. For example, the apertures 518a and 518b may be sized such that the corresponding fasteners 520a and 520b may be positioned in a plurality of positions along said length. By providing the apertures 518a and 518b at an angle relative to the bottom edge 508 of the panel and sizing the apertures 518a and 518b to receive fasteners 520a, 520b in a plurality of positions along the length of the apertures 518a, 518b the user may adjust the position and angle of the mandible shield 500 relative to the helmet shell 102.

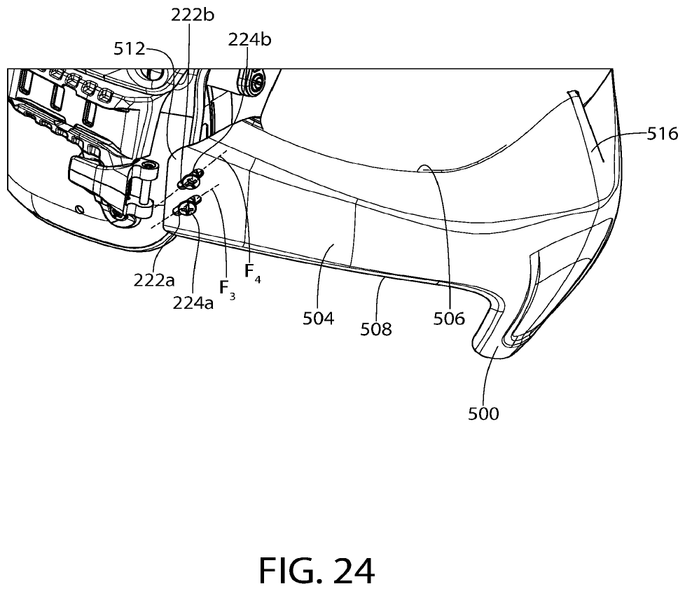

Referring to FIG. 24, the mandible shield 500 may include a second set of apertures including a third aperture 522a and fourth aperture 522b positioned on the right side 512 of panel 504. In some embodiments, the third aperture 522a and fourth aperture 522b may be similar to the first and second apertures 518a, 518b. In some embodiments, the third aperture 522a may extend along an axis F₃ and the fourth aperture 522b may extend along an axis F₄. In some embodiments, the axes F₃ and F₄ may be generally parallel and spaced from one another. In other embodiments, the axes F₃ and F₄ may not be generally parallel. In some embodiments, the axes F₃ and F₄ may each be at an acute angle relative to the bottom edge 508 of the panel. In some embodiments, axis F₃ and axis F₄ may be at generally the same angle relative to the bottom edge 508 of the panel 504.

Each aperture 522a and 522b may have a length extending along the corresponding axes F₃ and F₄. In some embodiments, each aperture 522a and 522b have generally the same length. In other embodiments, aperture 522a may have a length which is different than the length of aperture 522b. In some embodiments, the third aperture 522a and fourth aperture 522 are each configured to receive a corresponding fastener 524a and 524b for adjustably coupling the mandible shield 500 to the mounting rail 502. For example, the apertures 522a and 522b may be sized such that the corresponding fasteners 524a and 524b may be positioned in a plurality of positions along said length. By providing the apertures 522a and 522b at an angle relative to the bottom edge 508 of the panel and sizing the apertures 522a and 522b to receive fasteners 524a, 524b in a plurality of positions along the length of the apertures 518a, 518b the user may adjust the position and angle of the mandible shield 500 relative to the helmet shell 102.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “inwardly” and “outwardly” refer to directions toward and away from, respectively, the geometric center of the helmet. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. An adjustment mechanism comprising:
a housing including a sidewall extending circumferentially around a central axis, the sidewall defining an opening extending along the central axis and including a first aperture extending through the sidewall in a direction perpendicular to the central axis;
a receiving member coupled to the housing and rotatable about the central axis, the receiving member including a protrusion extending along the central axis, the protrusion including an aperture extending along an axis spaced from and parallel to the central axis;
a cable having a first end and a second end, the first end extending through the first aperture in the housing and the aperture in the receiving member and fixedly coupled to the receiving member, the second end fixed relative to the housing;
a knob coupled to the housing and rotatable about the central axis, the knob including a first protrusion configured to engage the protrusion of the receiving member such that rotation of the knob causes the receiving member to rotate; and
a locking element engaged with an interior surface of the sidewall of the housing, the locking element configured to be disengaged with the interior surface of the sidewall in response to a manual rotation of the knob by a user.

2. The adjustment mechanism of claim 1, wherein the locking element includes a biasing element engaged with the interior surface of the sidewall of the housing and configured to disengage from the interior surface of the sidewall in response to manual rotation of the knob by the user.

3. The adjustment mechanism of claim 2, wherein the biasing element is a spring coiled about the central axis, a first terminus extending inwardly toward the central axis, a second terminus, opposite the first terminus, extending from inwardly toward the central axis.

4. The adjustment mechanism of claim 3, wherein at least one of the first terminus and second terminus of the spring is configured to engage the first protrusion of the knob such that rotation of the knob causes the spring to compress.

5. The adjustment mechanism of claim 1 further comprising:
a base configured to receive at least one compressible comfort pad, the base comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side.

6. The adjustment mechanism of claim 5 further comprising:
a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism.

7. The adjustment mechanism of claim 5, wherein the base includes a flexible arm configured to couple the base to a helmet shell, wherein rotation of the knob causes the base to be displaced relative to the housing and causes the flexible arm to be elastically deformed.

8. An adjustment mechanism comprising:
a housing including a sidewall extending circumferentially around a central axis, the sidewall defining an opening extending along the central axis and including a first aperture extending through the sidewall in a direction perpendicular to the central axis;
a receiving member coupled to the housing and rotatable about the central axis, the receiving member including a protrusion extending along the central axis, the protrusion including an aperture extending along an axis spaced from and parallel to the central axis;
a cable having a first end and a second end, the first end extending through the first aperture in the housing and the aperture in the receiving member and fixedly coupled to the receiving member, the second end fixed relative to the housing;
a knob coupled to the housing and rotatable about the central axis, the knob including a first protrusion configured to engage the protrusion of the receiving member such that rotation of the knob causes the receiving member to rotate;
a locking element engaged with an interior surface of the sidewall of the housing, the locking element configured to be disengaged with the interior surface of the sidewall in response to a manual rotation of the knob by a user;
a base configured to receive at least one compressible comfort pad, the base comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side; and
a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism,
wherein the base includes a flexible arm configured to couple the base to a helmet shell, wherein rotation of the knob causes the base to be displaced relative to the housing and causes the flexible arm to be elastically deformed.

9. The adjustment mechanism of claim 8, wherein the locking element includes a biasing element engaged with the interior surface of the sidewall of the housing and configured to disengage from the interior surface of the sidewall in response to manual rotation of the knob by the user.

10. The adjustment mechanism of claim 9, wherein the biasing element is a spring coiled about the central axis, a first terminus extending inwardly toward the central axis, a second terminus, opposite the first terminus, extending from inwardly toward the central axis.

11. The adjustment mechanism of claim 10, wherein at least one of the first terminus and second terminus of the spring is configured to engage the first protrusion of the knob such that rotation of the knob causes the spring to compress.

12. An aviation helmet comprising:
a helmet shell having an outer surface and an inner surface; and
a base configured to be positioned proximate a nape of a user's neck when the user wears the helmet shell, the base adjustably coupled to the helmet shell by an adjustment mechanism, the adjustment mechanism comprising:
a housing fixedly coupled to the helmet shell and including a sidewall extending circumferentially around a central axis, the sidewall defining an opening extending along the central axis and including a first aperture extending through the sidewall in a direction perpendicular to the central axis;
a receiving member coupled to the housing and rotatable about the central axis, the receiving member including a protrusion extending along the central axis, the protrusion including an aperture extending along an axis spaced from and parallel to the central axis;
a cable coupled to the base and having a first end and a second end, the first end extending through the first aperture in the housing and the aperture in the receiving member and fixedly coupled to the receiving member, the second end fixedly coupled to the helmet shell;
a knob coupled to the housing and rotatable about the central axis, the knob including a first protrusion configured to engage the protrusion of the receiving member such that rotation of the knob causes the receiving member to rotate; and
a locking element engaged with an interior surface of the sidewall of the housing, the locking element configured to be disengaged with the interior surface of the sidewall in response to a manual rotation of the knob by a user.

13. The aviation helmet of claim 12, wherein the locking element includes a biasing element engaged with the interior surface of the sidewall of the housing and configured to disengage from the interior surface of the sidewall in response to manual rotation of the knob by the user.

14. The aviation helmet of claim 13, wherein the biasing element is a spring coiled about the central axis, a first terminus extending inwardly toward the central axis, a second terminus, opposite the first terminus, extending from inwardly toward the central axis.

15. The aviation helmet of claim 14, wherein at least one of the first terminus and second terminus of the spring is configured to engage the first protrusion of the knob such that rotation of the knob causes the spring to compress.

16. The aviation helmet of claim 12, wherein the base is configured to receive at least one compressible comfort pad, the base being comprised of a rigid material and having an outer surface, an inner surface and a protrusion extending outwardly from the outer surface, the outer surface being generally convex and the inner surface being generally concave, the protrusion defining an first aperture disposed on a first side of the protrusion and a second aperture disposed on a second side of the protrusion opposite the first side.

17. The aviation helmet of claim 16 further comprising:
a strap extending through the first aperture and the second aperture in the base, the strap configured to receive a fastener at a first end of the strap such that a position of the strap is fixed relative to the adjustment mechanism, the first strap having a second end opposite the first end, the second end looped around the cable of the adjustment mechanism.

18. The aviation helmet of claim 16, wherein the base includes a flexible arm coupling the base to the helmet shell, and wherein rotation of the knob causes the base to be displaced relative to the housing and causes the flexible arm to be elastically deformed.

19-34. (canceled)
